(19) United States
(12) Patent Application Publication
(10) Pub. No.: US 2001/0046464 A1
(43) Pub. Date: Nov. 29, 2001

(54) Sterilization device for foodstuff packaging covers

(75) Inventor: Ensio Mykkanen, Valkeakoski (FI)

Correspondence Address: FITCH EVEN TABIN AND FLANNERY, 120 SOUTH LA SALLE STREET, SUITE 1600, CHICAGO, IL, 606033406

(73) Assignee: UPM-Kymmene Corporation

(21) Appl. No.: 09/829,426

(22) Filed: Apr. 9, 2001

Related U.S. Application Data
(63) Continuation of application No. PCT/FI99/00406, filed on May 12, 1999.

(51) Int. Cl. A61L 2/18

(52) U.S. Cl. 422/302

(57) ABSTRACT

An apparatus for sterilizing lids for food packaging, comprising two sterilization wheels provided with gripping members and arranged to rotate in opposite directions inside a sterilization chamber, the said wheels being arranged to move to successive stop positions at stations provided at equal distances in the wall of the sterilization chamber, the functioning of the wheels at certain stations being as follows: at the take-up station of the first wheel, the gripping member is arranged to take the lid from a transfer device from outside the sterilization chamber: at the delivery station of the first wheel, the lid is arranged to move onto the second wheel while the gripping member of the second wheel is arranged to grip the other side of the lid at the take-up station of the second wheel, and at the delivery station of the second wheel, the sterilized lid is arranged to move into a food container outside the sterilization chamber.

The object of this invention is an apparatus for sterilising lids for food containers. The apparatus comprises a sterilisation chamber in the wall of which there are inlets for gaseous or gas/liquid mixtures and a sterilisation wheel with gripping members.

Known devices for sterilising lids for food containers function by one or other of the following principles: a) The containers are filled and packed in a clean atmosphere and the food container with the contents is sterilised by applying heat or hot water. b) The food is sterilised and packed into containers that have been sterilised beforehand, and the containers are sealed with a sterile lid in a sterile atmosphere. The method mentioned in item b) has several advantages compared to that mentioned in item a).

A typical method according to item b) for sterilising lids for single portion containers is presented in U.S. Pat. No. 4,981,649. A disadvantage of known methods is that the structure of the devices is rather complicated. For example, in the said US-patent, the wall of the outer surface of the sterilisation wheel has sealed spaces which have a plurality of gripping members, and after the wheel has revolved and arrived at a feed mouth for the sterilising agent, the sterilising agent spreads into the space. The sterilising of the same side of the lid is not possible without moving the sucking members holding the lid during the sterilisation process. Similarly, effective sterilisation of the lid on the opposite side to the direction of feed of the sterilisation agent cannot be fully guaranteed.

The purpose of this invention is to present an apparatus, the structure of which is simple and reliable and which provides means for sterilising lids for food containers in an inexpensive and effective manner. In order to achieve this, an apparatus according to the invention is mainly characterised in what is presented in the characterising part of the attached claim 1.

An apparatus according to the invention provides means for sterilising the whole surface of a lid one side at a time without moving the gripping members. In this manner, the whole surface is completely sterilised, as there are no obstacles in the way of the sterilising agent feed mouth. The apparatus comprises two sterilisation wheels rotating in opposite directions and in which one side of the lid is treated at a time from the side of the outer circumference.

The invention will be described in the following with reference to the accompanying drawing, in which

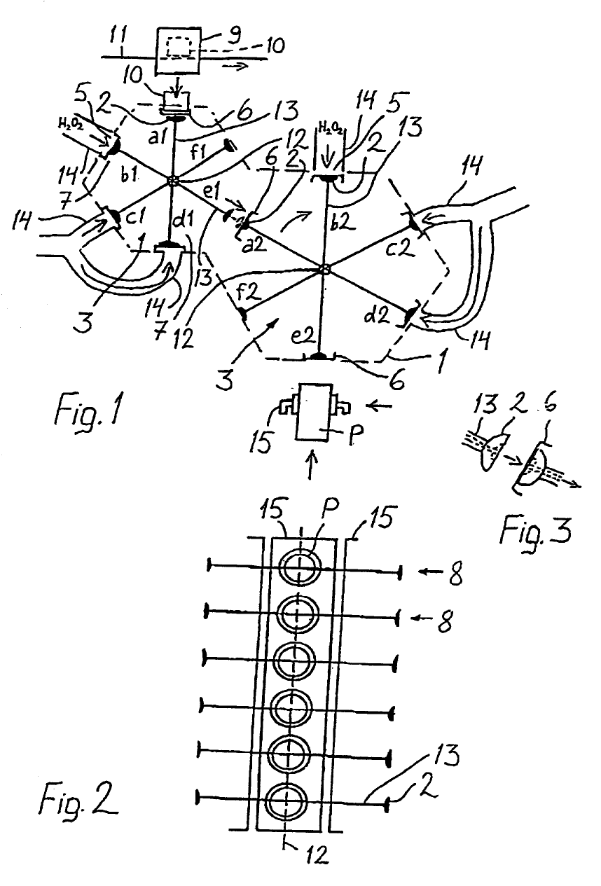

FIG. 1 shows the side view of the apparatus according to the invention, and

FIG. 2 shows several sterilisation units arranged on the same axle, and

FIG. 3 illustrates an advantageous means for transferring the lid.

FIG. 1 shows the forming and sterilisation of a lid in a sterilisation and filling line for food containers. At first, a deep drawing punch 9 cuts lid blanks from band 11 and a deep drawing mandrel 10 presses the lid 6 down on a gripping member. At the same time, the edges of the lid fold upwards, because the mandrel forces the lid blank through a hole which is smaller than the surface of the blank. This upright edge is later utilised in sealing the lid into the body of the container in a manner in which the upper edge of the container's body is folded inwards over the upright edge of the lid 6.

The actual sterilising apparatus comprises two sterilisation wheels 3 provided with gripping members 2, arranged to rotate in opposite directions inside a sterilisation chamber 1, the said sterilisation wheels being arranged to rotate forward to successive stop positions at stations provided at equal distances, i.e. at certain angular distances from each other, in the wall of the sterilisation chamber, i.e., the rotation takes place sequentially with the help of a motor connected to the rotation axle 12. The gripping member 2 may, for example, be a known suction pad structure, to which can be connected vacuum suction or equivalent by means of suction ducts, such as suction hoses or equivalents (not shown), brought through the axle 12. On the same axles 12 of the sterilisation wheels 3 there can be several sterilisation units 8, each of which is formed of rods 13 provided at angular distances, projecting radially from the axle, each rod having a comparable gripping member 2 at its end.

The sterilisation chamber is essentially closed off from its surroundings, manufactured from e.g. transparent material, and in the walls of the chamber opposite the gripping members are the mouths 5 of ducts 14 connected to the source of medium. The ducts are supplied with feeding of the media necessary for the process of sterilising the lid. The flow coming from the mouths 5 is radially directed inwards toward the outer surface of the lid 6 held in the gripping member 2. Inside the sterilisation chamber 1 there are local outlets 7, from which the exhaust air is sucked in the corresponding sector, in order to discharge the surplus of the substances fed into the chamber. The location of the local outlets is not presented in great detail but they can be placed close to the gripping members. Moreover, overpressure is maintained in the sterilisation chamber 1 in relation to the outside air, in order to prevent the entry of contaminating substances from the outside.

The functioning at the stations is as follows: at a1, the gripping member 2 of the first wheel 3 takes the lid 6 from a transfer device from outside the sterilisation chamber in such a manner that the upright edge of the lid is left facing away from the gripping member, e.g. directly away from the deep drawing mandrel 10, while the gripping member 2 points upwards against the thrust motion of the deep drawing mandrel 10, at b1 a blast of hot sterilising agent, e.g. hydrogen peroxide in a suitable form, such as hydrogen peroxide spray, is blown toward the surface of the lid, at c1 and d1 a blast of hot air is blown to activate and dry the sterilising agent, and finally, after the lowest point of the rotational movement at the delivery station e1, the lid 6 is transferred onto the second wheel, whereby the gripping member 2 of the second wheel 3 grips the other side of the lid at the take-up station a2, i.e., the upright edge of the lid is on the side of the gripping member and the side that forms the inner surface of the lid inside the container faces outwards on the circumference of the wheel. At this stage, the gripping members 2 face one another in such a manner that the rod 13 of the first sterilisation wheel points obliquely downwards and the rod 13 of the second sterilisation wheel points obliquely upwards.

The transfer can occur, for example, as a thrust motion of the gripping member 2, in order to carry out which motion, the supporting rod 13 of the gripping member 2 may be reciprocating, e.g. a telescopic structure, as is presented in FIG. 2. According to an advantageous embodiment, the transfer is carried out in such a manner that the gripping member 2 generates an impulse e.g. with the help of a blast from the gripping member, the lid 6 remaining in place, and thus, the lid 6 flies a short distance to the opposite gripping member 2 of the second wheel 3, the said gripping member now being provided with suction, which attaches the lid 6 to it. If the gripping members 2 have been arranged to hold the lids 6 in place with suction, it is easy to change the suction into a blast with the help of a valve in the duct between the underpressure source and the gripping member, in order to transfer the lid. Thus, the gripping members 2 are at a fixed distance from the rotation axle of the sterilisation wheel at every station, also at the delivery station e1. The above-mentioned transfer embodiment is presented in FIG. 3. If the gripping members are close enough to one another at their stations e1 and e2, it is possible to arrange the transfer in such a manner that suction is cut off from the gripping member of the first sterilisation wheel, in which case suction in the gripping member 2 of the second sterilisation wheel can be sufficient to draw the lid onto the second sterilisation wheel 3, and if not, suction can be increased at this station a2.

The function of the second sterilisation wheel 3 at the various stations is as follows, while the gripping member 2 moves via the highest point of the rotational movement to a position in which the rod 13 points downwards in order to deliver the lid into the container: at b2 a blast of hot sterilising agent is blown onto one side of the lid 6, which has now moved outwards to the circumference of the sterilisation wheel, at c2 and d2 a blast of hot air is blown, and at e2 the sterilised lid is transferred into a food container P in such a manner that the upper edge of the lid 6 remains in the upper part of the container against the inner wall of the container body. The stages ax−ex (x=1 or 2) in both wheels are repeated intermittently in the same order. Stations f1 and f2 are empty, and stations b1, b2, c1, c2, d1 and d2 have in the circumferential walls of the sterilisation chamber, ducts and equivalent feed mouths which open against the lid, or nozzles for blowing gaseous or gas/liquid mixtures. The delivery stations e1 and e2 of both wheels are arranged so that their distance from the axle of the sterilisation wheel can be adjusted, or the delivery station e2 of the latter wheel can be at a fixed distance from the axle, while the container P, open from above, is lifted up to meet the lid 6, and it is not necessary to lower the lid down.

When the lid 6 is placed inside the container above the contents, the container is transferred from underneath the delivery station e2 into handling units, where the lid 6 is finally sealed onto the container.

FIG. 2 shows the second sterilisation wheel 3 from above, and below it, also the conveyor for the containers P, which is formed of consecutive beams 15 in which there are adjacent places for carrying several containers open from above side by side and step by step between the different stages in the sterilisation and filling line and below the delivery station e2 of the second sterilisation wheel 3. It can be seen from the figure that on the axle 12 of the wheel 3, there can be several adjacent sterilisation units 8 comprising gripping members radially projecting outwards. The first wheel 3 naturally has an equal number of sterilisation units next to each other.

It is clear that the different embodiments of the invention are not limited to the above-mentioned example, but they can vary within the scope of the attached claims. The gripping members can function according to different principles, and the function and/or the composition of the substance blown onto the lid 6 at different stations can be changed. The sterilisation chamber 1 can be divided into suitable parts with different partition walls, e.g. the handling stations can be isolated into their own sectors so that the gripping members are able to move from one sector to another. The sterilisation chamber 1 can also be such that it can accommodate both the sterilisation wheels inside it.

The invention is intended for sterilising lids 6, especially in the kind of sterilisation and filling lines in which the container body that has been sterilised beforehand has been filled with foodstuff, most generally with a beverage, and the filled body is carried in the conveyor under the delivery station e2, after which the lid 6 and the body are joined together. At this stage, also the feeding of a protective gas or another suitable gas composition into the upper part of the container above the contents takes place in order to replace the gas composition which was there first.

The invention is not limited to the material for lids or containers, but the said invention can be applied, for example, to liquid packaging board, especially such containers where the paperboard blank is wrapped into a cylindrical, conical or other closed structure in horizontal section, and where the end that is open from above, of the body which is closed with a bottom lid from below, is finally closed with a separate cover piece. Similarly, the term “lid” in this respect is to be understood as such a part that is joined to an otherwise ready and filled container in order to fully close it, and it can comprise an opening, e.g. a hole covered with a tear strip, meant for emptying the container, or it can be an intact bottom lid which, nevertheless, forms a lid at the closing stage as shown in FIG. 1, and at the stage of use, the end member functioning as a lid is the bottom in connection with filling and closing.

1. An apparatus for sterilising lids for food packaging, comprising a sterilisation wheel provided with gripping members and arranged to rotate inside a sterilisation chamber, the said wheel being arranged to move to successive stop positions at stations provided at equal distances on the course of the rotary movement, wherein the said apparatus comprising two sterilisation wheels being arranged to rotate, the functioning of the said wheels at certain stations being as follows: at the take-up station of the first wheel, the gripping member is arranged to take the lid from a transfer device from outside the sterilisation chamber, at the delivery station of the first wheel, the lid is arranged to move onto the second wheel, while the gripping member of the second wheel is arranged to grip the other side of the lid at the take-up station of the second wheel, and at the delivery station of the second wheel, the sterilised lid is arranged to move into a food container outside the sterilisation chamber.

2. An apparatus as claimed in claim 1, wherein into stations between the take-up stations and delivery stations in both wheels is arranged a blast of hot sterilising agent onto the lid and a hot air blast to activate and dry the sterilising agent.

3. An apparatus as claimed in claims 1 or 2, wherein in both wheels, the stages are repeated sequentially in the same order.

4. An apparatus, as claimed in claim 1, wherein the gripping member is a suction pad or an equivalent member functioning by means of suction.

5. An apparatus as claimed in claim 3, wherein at a station between the take-up and delivery stations there is at least one mouth in the wall of the sterilisation chamber, the said mouth being connected to a source of medium.

6. An apparatus as claimed in claim 5, wherein on the same axle of a sterilisation wheel there is a plurality of adjacent sterilisation units.

7. An apparatus as claimed in claim 6, wherein in comparison with the outside air there is an overpresssure in the sterilisation chamber.

8. An apparatus as claimed in claim 7, wherein the sterilisation wheels are arranged to rotate in the same sterilisation chamber.

9. An apparatus as claimed in claim 8, wherein at the delivery station of the first sterilisation wheel, the distance between the gripping member and the axle of the said sterilisation wheel remains fixed.

10. An apparatus as claimed in claim 9, wherein at the delivery station of the first sterilisation wheel, the gripping member is arranged to generate an impulse to the lid in order to move the lid to the gripping member of the second sterilisation wheel which is located opposite the first gripping member.
